(19) United States
(12) Patent Application Publication
(10) Pub. No.: US 2022/0227828 A1
(43) Pub. Date: Jul. 21, 2022

(54) METHOD FOR OVEREXPRESSING IL-15 IN PORCINE SKELETAL MYOBLASTS AND USE THEREOF

(71) Applicant: Institute of Animal Sciences of Chinese Academy of Agricultural Sciences, Beijing (CN)

(72) Inventors: Yue Hao, Beijing (CN); Xianhong Gu, Beijing (CN); Mingjie Xing, Beijing (CN)

(21) Appl. No.: 17/561,378

(22) Filed: Dec. 23, 2021

(30) Foreign Application Priority Data
Jan. 19, 2021 (CN) 202110066701.5

(51) Int. Cl. C07K 14/54; C12N 15/86; C12N 5/077

(52) U.S. Cl. CPC C07K 14/5443; C12N 2740/15043; C12N 5/0658; C12N 15/86

(57) ABSTRACT

The present disclosure relates to the field of genetic engineering, in particular to a method for overexpressing IL-15 in porcine skeletal myoblasts and use thereof. In the present disclosure, the porcine IL-15 gene is transferred into the lentiviral vector and then the lentiviral vector is transferred into the porcine skeletal myoblasts, so that IL-15 is successfully overexpressed in the porcine skeletal myoblasts. The method for overexpressing IL-15 provided by the present disclosure solves the problem that IL-15 needs to be added multiple times and the steps are complicated in the traditional research process. The present disclosure demonstrates that overexpression of IL-15 has a regulating effect on proliferation, cell apoptosis, anti-oxidation capacity of the porcine skeletal myoblasts, which charts a course for further study of IL-15.

### CROSS REFERENCE TO RELATED APPLICATION

This patent application claims the benefit and priority of Chinese Patent Application No. 202110066701.5 filed with China National Intellectual Property Administration on Jan. 19, 2021, the disclosure of which is incorporated by reference herein in its entirety as part of the present application.

### TECHNICAL FIELD

The present disclosure relates to the field of genetic engineering, in particular to a method for overexpressing IL-15 in porcine skeletal myoblasts and use thereof.

### BACKGROUND

With the increase in the intensification degree of China's animal husbandry industry, especially modern breeding industry, pigs are affected by more and more stress factors during growth, fattening and slaughter, causing changes in nutrient distribution and free radical metabolism in the pig body. Stress will break the balance of oxidation and anti-oxidation in the body, leading to a sharp increase of free radicals in the body, which causes irreversible oxidative damage to many important biological macromolecules in the body and further destruction of cell structure and function, and ultimately affects animal health and meat quality (Kerth, 2013).

Interleukin-15 (IL-15) , discovered in 1994, was a pleiotropic cytokine capable of regulating the proliferation, differentiation, maintenance and activity of a variety of cells [2]. IL-15 is also an important muscle factor secreted by skeletal muscle and plays an important role in regulating metabolism of skeletal muscle and energy balance. It can regulate the metabolism of muscle and adipose tissue in the modes of endocrine and paracrine, thereby maintaining the homeostasis of the whole body. There are more and more researches on IL-15. However, prior studies mainly involved the increased expression of IL-15 in the cells by using the method of directly adding IL-15. Because IL-15 has a short half-life, it is difficult to achieve the desired effect in a single treatment, there is need for multiple addition of IL-15, the operation is cumbersome, and the research cost is increased. And there is no report on the effect of IL-15 on porcine skeletal myoblasts.

### SUMMARY

The present disclosure provides a method for overexpressing IL-15 in porcine skeletal myoblasts and use thereof to solve the above-mentioned problems in the prior art. Overexpressing IL-15 in porcine skeletal myoblasts not only reduces the research cost, but also simplifies the operation steps. The present disclosure proves that overexpression of IL-15 has a regulatory effect on among the others the cell cycle and apoptosis rate of porcine skeletal myoblasts, and lays a foundation for further research on the effect of IL-15 on livestock and poultry meat.

In order to achieve the above objective, the present disclosure provides the following technical solutions.

In a first aspect, there is provided a method for overexpressing IL-15 in porcine skeletal myoblasts, comprising transforming IL-15 into a lentiviral vector, then transforming the lentiviral vector into porcine skeletal myoblasts to promote the porcine skeletal myoblasts to overexpress IL-15.

In an embodiment of the present disclosure, the lentiviral vector is GV492 lentiviral expression vector.

In a second aspect, there is provided the use of overexpression of IL-15 in reducing apoptosis of porcine skeletal myoblasts.

In a third aspect, there is provided the use of overexpression of IL-15 in enhancing the ability of porcine skeletal myoblasts to resist oxidative damages.

In a fourth aspect, there is provided the use of overexpression of IL-15 in promoting division and proliferation of porcine skeletal myoblasts.

The present disclosure provides a method for overexpressing IL-15 in porcine skeletal myoblasts and its uses. In the present disclosure, lentiviral vector is used to transfer the gene of porcine IL-15 into porcine skeletal myoblasts, and the overexpressed of IL-15 in porcine skeletal myoblasts can be proven by detecting the mRNA and protein expression level. In the present disclosure, the gene of IL-15 is overexpressed directly in the porcine skeletal myoblasts, which addresses the problems of multiple additions and cumbersome operations in the treatment of IL-15 in prior studies, and lays a foundation for further study of the effect of IL-15 on meat.

In the present disclosure, it is proven through experiments that IL-15 is capable of promoting the division and proliferation of porcine skeletal muscle, enhancing its ability to resist oxidative damage and reducing the rate of cell apoptosis. There is also provided the use of overexpression of IL-15, which charts a course for further research.

### BRIEF DESCRIPTION OF THE DRAWINGS

FIG. 1 is a map of vector GV492 in Example 1.

FIG. 2 is an electrophoregram of the vector after digestion in Example 1, wherein Lane 1 is a 10 kb Marker (from top to bottom: 10 kb, 8 kb, 6 kb, 5 kb, 4 kb, 3.5 kb, 3 kb, 2.5 kb, 2 kb, 1.5 kb, 1 kb, 750 bp, 500 bp, and 250 bp, respectively), lane 2 is the digested product of the vector, and lane 3 is the undigested vector.

FIG. 3 is an electrophoregram of the target gene fragment in Example 1, wherein, the left band represents the Marker (5 kb, 3 kb, 2 kb, 1.5 kb, 1 Kb, 750 bp, 500 bp, 250 bp, and 100 bp, respectively), and the right bands represent PCR products.

FIG. 4 is an electrophoregram for PCR of the transformant in Example 1, wherein lane 1 represents the negative control (ddH₂O), lane 2 represents the negative control (unloaded self-linked control group), lane 3 represents the positive control (GAPDH), lane 4 represents the Marker (from top to bottom: 5 kb, 3 kb, 2 kb, 1.5 kb, 1 Kb, 750 bp, 500 bp, 250 bp, and 100 bp, respectively), lanes 5-12 represent transformants 1-8, respectively.

FIG. 5 is a diagram showing the results for sequence alignment of positive clones in Example 1.

FIG. 6 is a diagram showing the results for fluorescence observation of cells after transfection in Example 1.

FIG. 7 is an electrophoregram of the cells transfected in Example 1 by Western Blot, wherein lane 1 represents the positive control, lane 2 represents the negative control, and lane 3 represents the sample obtained from transinfection of plasmid for the target gene by 293T cells. And the primary antibody used in the electrophoresis was a Flag protein that encodes a hydrophilic peptide consisting of eight amino acids (DYKDDDDK, SEQ ID NO: 9). The secondary antibody used in the electrophoresis was goat anti-mouse IgG (H+L), HRP.

FIG. 8 is a diagram of porcine skeletal muscle satellite cells isolated and cultured in Example 2.

FIG. 9 is a diagram of the induced and differentiated porcine skeletal myoblasts in Example 2.

FIG. 10 is diagram showing the results for immunofluorescence assay, wherein it is shown from top to bottom the porcine skeletal muscle satellite cells, differentiated porcine skeletal myoblasts, porcine skeletal myoblasts transfected with control lentivirus, and porcine skeletal myoblasts transfected with overexpression lentivirus.

FIG. 11 is a diagram of results for cell cycle detection in Example 2.

FIG. 12 is a diagram showing the detection of apoptosis by flow cytometry in Example 2.

FIG. 13 is a diagram of showing the detection of apoptosis rate of porcine skeletal myoblasts by flow cytometry in Example 2;

FIG. 14 is a diagram showing the comparison of gene expression levels of IL-15 in porcine skeletal myoblasts in Example 2.

FIG. 15 is a diagram showing the comparison of protein expression levels of IL-15 in porcine skeletal myoblasts in Example 2.

FIG. 16 shows the effect of H202 on cell viability in Example 3.

FIG. 17 is a diagram showing the detection results of cell cycle under oxidative stress conditions in Example 3.

FIG. 18 is a diagram of flow cytometry of cell apoptosis under oxidative stress conditions in Example 3.

FIG. 19 is a diagram showing the detection of cell apoptosis rate by flow cytometry under oxidative stress conditions in Example 3.

FIG. 20 is the results for detection of cell apoptosis rate by Western Blot under oxidative stress conditions in Example 3.

FIG. 21 is a diagram showing the results for ROS detection of the negative group by flow cytometry in Example 3.

FIG. 22 is a diagram showing the results for ROS detection of the blank cell plus H₂O₂ group by flow cytometry in Example 3.

FIG. 23 is a diagram showing the results for ROS detection of virus transfected with the control group+hydrogen peroxide group by flow cytometry in Example 3.

FIG. 24 is a diagram showing the results for ROS detection of virus transfected overexpressing IL-15 with+hydrogen peroxide group in Example 3.

FIG. 25 is a diagram showing the results for ROS detection of the cells in Example 3.

FIG. 26 is a diagram showing the results of IL-15 secretion by cells in Example 3.

### DETAILED DESCRIPTION OF THE EMBODIMENTS

Various exemplary embodiments of the present disclosure will now be described in detail. The detailed description should not be considered as a limitation to the present disclosure, but should be understood as a more detailed description of certain aspects, characteristics, and embodiments of the present disclosure.

Unless otherwise specified, all technical and scientific terms used herein have the same meaning as commonly understood by those skilled in the art in the field of the present disclosure. Although the present disclosure only describes preferred methods and materials, any methods and materials similar or equivalent to those described herein can also be used in the practice or testing of the present disclosure.

Without departing from the scope or spirit of the present disclosure, various modifications and variations can be made to the specific embodiments of the present disclosure description, which will be apparent to those skilled in the art. Other embodiments derived from the description of the present disclosure will be apparent to the skilled person. The specification and examples of this disclosure are merely exemplary.

Unless otherwise specified, the materials and reagents used in the following examples can be available from commercial sources. Unless otherwise specified, the experimental methods used are all conventional experimental methods in the field.

### Example 1

Preparation of Lentivirus for Overexpressing Porcine Gene IL-15

The sequence of porcine gene IL-15 was queried in NCBI, and the accession number was NM_214390.

1. Construction of Overexpression Lentiviral Vector

1. Materials and Methods

1.1 Main Reagents

Main reagents are shown in Table 1.

TABLE 1
Main reagent information

| Reagent name | Reagent source | Model |
|---|---|---|
| 1kp DNA ladder Marker | Fermentas Inc. | #SM0311 |
| 250bp DNA ladder Marker | Shanghai Generay | DL250+, 100T |
| | Biotech Co., Ltd | |
| Agarose | Beijing SBS | GA4-100 |
| | Genetech Compay | |
| In-Fusion ™ PCR | clontech | 639626 |
| Cloning Kit | | |
| Taq polymerase | SinoBio | E001-02B |
| dNTP | Takara | D4030A |
| Primer | Shanghai Generay | |
| | Biotech Co., Ltd. | |
| Restriction endonuclease | NEB | |
| Plasmid extraction kit | Promega | A1460 |
| Agarose gel DNA | TIANGEN BIOTECH | DP209-03 |
| recovery kit | (BEIJING) CO., LTD. | |

1.3 Main Instruments And Equipment

Main instruments and equipment are shown in Table 2.

TABLE 2
Main instrument information

| Equipment name | Reagent source | Model |
|---|---|---|
| PCR instrument | Applied Biosystems | 2720 thermal cycler |
| Positive clone | Shanghai Meiji Biotech | ABI3730 |
| Sequencer | | |
| Constant voltage DNA | BioRad | |
| electrophoresis | | |
| apparatus | | |
| Gel imager | Shanghai Tanon Technology | |
| | Co., Ltd. | |
| Bacteria shaker | Taicang Hualida Laboratory | HI-9211K |
| | Equipment Co., Ltd | |
| Bacteria incubator | Shanghai Yiheng Scientific | |
| | Equipment Co., Ltd | |
| Gilson pipette | Gilson | |
| High-speed centrifuge | Hitachi | TGL-16G-A |
| Disposable disc | Hunan Changsha Tiandiren | |
| | biotechnology Co. Ltd. | |
| 1L flask | Jintan Jingbo Experimental | 1111.1115 |
| | Instrument Factory | |
| 50 ml polypropylene | Shanghai Wuhua Chemical | |
| tube | Co., Ltd. | |

2. Digestion of Vector

2.1 Vector Information

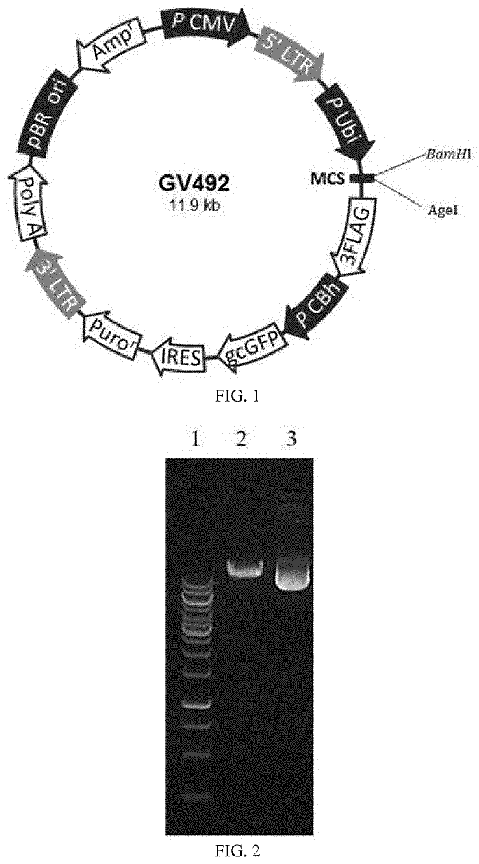

The name of the vector used is GV492, and the sequence of elements: Ubi-MCS-3FLAG-CBh-gcGFP-IRES-puromycin, which contains two cloning sites BamHI/AgeI. The map of the vector is shown in FIG. 1. The control empty vector number is CON335.

2.2 Results of Restriction Digestion 1601 Plasmids extracted from bacteria have different migration rates due to different conformations such as supercoil, open loop, linearity, and present bands of different sizes in agarose gel electrophoresis. Therefore, the electrophoresis bands of plasmids can only be used as a reference for determining the molecular weight of plasmids, and it cannot be used as a basis for accurate determination. After the plasmids were subjected to single digestion, it showed a uniform electrophoretic band. At this time, comparison to the Marker could be used to determine its molecular mass. The PCR result of the vector after digestion is shown in FIG. 2. It can be seen that the vector after single digestion formed a uniform band. When compared with the Marker, the molecular weight of the band was the same as recorded in the vector map, which proved successful digestion of the vector.

3. Acquisition of Target Gene Fragments

3.1 Primer Information

Full gene synthesis was performed according to the sequence of the target gene, and the target gene was amplified by PCR according to the manual of the vector (Overexpression Lentiviral Vector Package and Construction Manual, https://www.genechem.com.cn/index/supports/download.html). Information on primers for PCR is shown in Table 3. It is shown that the primer contained a base pair exchange, and restriction digestion sites. The primer further contained partial sequence at the 5′-end of the target gene, which was used to tag the target gene by PCR.

TABLE 3
PCR primer information (SEQ ID NO.1-SEQ ID NO.2)

| ID | Sequence |
|---|---|
| IL15(44657-6)-P1 | AGGTCGCTCTAGAGGATCCCGCCACCATGAGA |
| | ATTTTGAAACCATGTTTG |
| IL15(44657-6)-P2 | TCCTTGTAGTCCATACCAGAAGGGTTGATGAA |
| | CATTTGC |

3.2 PCR Results

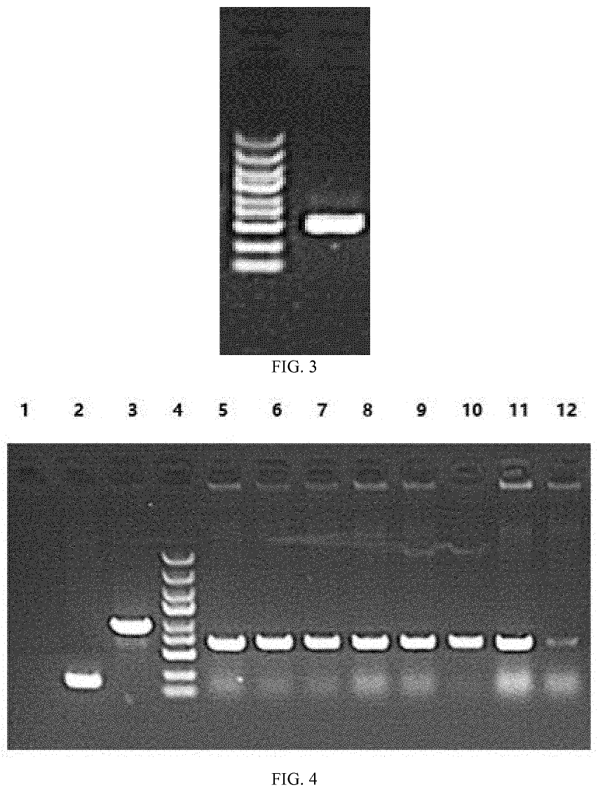

The electrophoregram for PCR is shown in FIG. 3, and the product is about 530 bp in size. It was proven that the target gene fragment was successfully obtained.

4. Construction of Recombinant Plasmid

The resulting PCR product of the target gene was exchanged into the linearized expression vector for PCR identification, according to the vector specification (overexpression lentiviral vector package and construction manual, https://www.genechem.com.cn/index/supports/download.html). The information on primers for PCR identification is shown in Table 4.

TABLE 4
Primers for PCR identification
(SEQ ID NO.3-SEQ ID NO.4)

| ID | Sequence |
|---|---|
| Ubi-F | GGGTCAATATGTAATTTTCAGTG |
| FLAG-R-2 | CCTTATAGTCCTTATCATCGTC |

The results for PCR identification is shown in FIG. 4, the PCR product of positive transformants have a size of 669 bp, and the PCR product of negative transformants has a size of 185 bp.

The positive transformants were subjected to sequencing and alignment, and the alignment results (FIG. 5) showed that the overexpression lentiviral vector was successfully constructed.

2. Detection of IL-15 Expression

The successfully constructed lentiviral vector was used to transfect 293T cells, the cell fluorescence was observed with a fluorescence microscope, and the expression of IL-15 was detected by Western Blot. The size of the target gene fusion protein was about 20 kD.

1. Materials and methods

The target cells to be transfected were 293T cells cultured in DMEM medium (containing 10% fetal bovine serum). The antibody information used is shown in Table 5.

TABLE 5
Antibody information

| | | Antibody | | Product | Dilution |
|---|---|---|---|---|---|
| No. | Antibody | name | Manufacturer | No. | ratio |
| 1 | Primary | FLAG | Sigma | F1804 | 1:2000 |
| | antibody | | | | |
| 2 | Secondary | Mouse | santa-cruz | sc-2005 | 1:10000 |
| | antibody | | | | |

The above information comes from the antibody instructions, including the antibody dilution ratio (recommended dilution ratio in the instructions), and positive control samples (recommended positive control in the instructions).

2. Experimental Method

2.1 Extraction of Cell Proteins

The RIPA protein extraction reagent was pre-cooled and protease inhibitors were added in the protein extraction reagent. 0.1M PMSF mother liquid was added before the start of protein extraction, the final concentration of PMSF was 1 mM. Cell count: The cells was added to 300 μL of lysis buffer in a number of 1×10⁷ and blown with the pipette tip to suspend the cells. The cells were incubated on ice for 20 min after the completion of suspension. And centrifugation was conducted for 20 min at 4° C. and 13000 rpm. After the completion of the centrifugation, the supernatant was dispended for further measurement.

2.2 Quantification by BCA Protein Method

BCA working solution (solution A: solution B=50:1) was prepared, and respective BSA extraction standard. The sample was diluted with PBS.

The sample and the BCA working solution was mixed in a ratio of 1:8. After being mixed uniformly, the mixture was incubated at 37° C. for 30 min or at room temperature for 60 min. The OD value was measured with a 570 nm-wavelength filter in the enzyme-labeled instrument.

2.3 Adjustment of Protein Concentration

The protein concentration was adjusted with RIPA and 5× reducing sample buffer was added to a sample final concentration of 2-4 mg/ml (determined with respect to different samples). The mixture was boiled for denaturation for 5 min.

2.4 Western Blot Experiment

(1) 10% of separating gel was based on the molecular weight of the target protein, and the concentration of the concentrated gel was 5%.

(2) 20 μg of protein sample was loaded, a WB standard (SURVIVIN-3FLAG-GFP, molecular weight: 48 kD) was used as the positive control, and the 293T cells were used as the negative control.

(3) Electrophoresis conditions: concentrated gel, constant voltage at 90V for about 20 min; separating gel constant voltage 160V, and the time to stop the electrophoresis was determined by pre-stained the protein Marker.

(4) Wet transfer method: membrane transfer conditions: 300 mA of constant electric current; 0.45 μM pore size NC membrane, membrane transfer time 2.5 h. After completion of membrane transfer, the membrane was stained with the staining reagent Ponceau, membrane transfer effect was observed, and the lanes were marked at the same time.

(5) Blocking: the membrane was completely immersed in 3% BSA-TBST and gently shaken at room temperature for 30 minutes.

(6) Primary antibody incubation: the primary antibody was diluted with 3% BSA-TBST, incubated at room temperature for 10 minutes, and allowed to stand overnight at 4° C. And the membrane was taken out from 4° C. incubator the next day, and then incubated at room temperature for 30 minutes. Membrane washing: the membrane was washed 5 times with TBST, 3 min per wash.

(7) Secondary antibody incubation: the secondary antibody goat anti-mouse IgG (H+L) HRP was diluted with 5% skimmed milk powder-TBST in a ratio of 1:10000, and the diluted solution was gently shaken at room temperature for 40 minutes. Membrane wash: the membrane was washed with TBST 6 times, 3min per wash.

(8) Color development by ECL method combined with X-ray film: after ECL was added to the film, the reaction was carried out for 3-5 min, the film was exposed to light for 10 s-5 min (the exposure time was adjusted based on the light intensity), developed for 2 min, and fixed.

2.5 Detection of Virus Titer

The virus titer was measured using a fluorescence method, and the measured virus titer was 2.5E+9.

3. Experimental Results

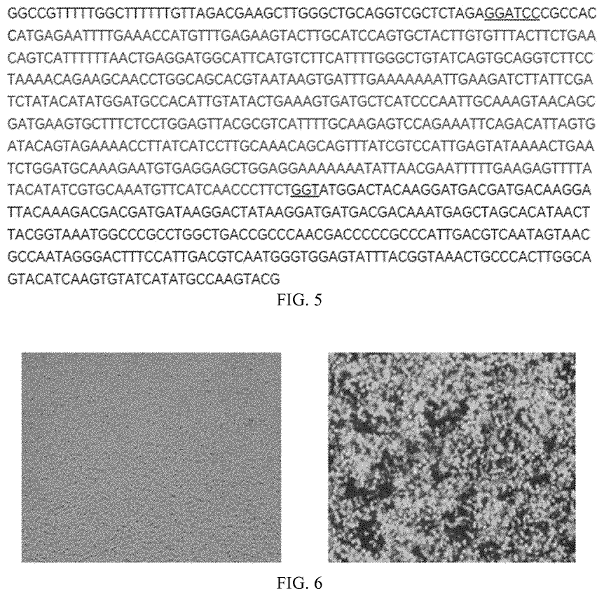

The result of cell fluorescence observation after transfection is shown in FIG. 6. It can be seen that notable fluorescence could be observed in the cells after transfection, indicating that the target plasmid was normally transfected and the expression of the fluorescent marker gene in the target plasmid was normal.

The results of Western Blot electrophoresis are shown in FIG. 7. It can be seen that there was an obvious characteristic band at 20 kD, and the size was consistent with the target gene fusion protein, which proved the successful overexpression of the target gene.

### Example 2

Overexpression of IL-15 in Porcine Skeletal Myoblasts

1. Materials and Equipment

The required experimental equipment is shown in Table 6, and the materials and reagents are shown in Table 7.

TABLE 6
Experimental instrument information

| | | Specification |
|---|---|---|
| Equipment name | Manufacturer | and model |
| Biological safety cabinet | Jinan BIOBASE Co., Ltd. | BSC-1500IIA2-X |
| CO₂ cell incubator | Shanghai Boxun Industry & | BC-J160S |
| | Commerce Co., Ltd. | |
| Fluorescence inverted | Nikon | DS-Ri2 |
| microscope | | |
| High-speed refrigerated | Thermo Fisher | Multifuge X1R |
| centrifuge | | |
| Electric heating constant | Shanghai Jing Hong | DHG-9123A |
| temperature blast drying | Laboratory | |
| oven | Instrument Co., Ltd. | |
| Electric heating constant | Shanghai Jing | DK-2B |
| temperature oscillating | Hong Laboratory | |
| sink | Instrument Co., Ltd. | |
| Flow cytomerters | Miltenyi Biotec | 130-092-197 |

TABLE 7
Experimental materials and reagent information

| | | Specification/ |
|---|---|---|
| | | Product |
| Reagents and consumables | Manufacturer | number |
| T25 cell culture flask | Coming | 430639 |
| Hemocytometer | Marienfeld | Neubauer |
| | | improved |
| Special round coverslips | Solar bio | YA0350 |
| for 24-well plate | | |
| Cell culture plate | Shanghai WHB | |
| | Scientific Co., Ltd. | |
| Fetal bovine serum | Gibico | 1414426 |
| Special culture medium | Shanghai iCell | Primed-iCell- |
| for skeletal muscle cells | Bioscience Inc. | 018 |
| 0.25% trypsin (containing | Gibico | 1734858 |
| 0.02% EDTA) | | |
| Paraformaldehyde (PFA) | Solarbio | P1110 |
| DAPI | Solarbio | C0060 |
| Triton X-100 | Solarbio | T8200 |
| Goat serum | Solarbio | SL038 |
| α-SM | Abeam | Ab124964 |
| CoraLite594-conjugated | Proteintech | SA00013-4 |
| goat anti-Rabbit | | |
| IgG (11 + L) | | |
| Fluoromount-G | SourthernBiotech | 0100-01 |
| fluorescence Mounting | | |
| agent | | |
| LV-IL15 | GeneChem Co., Ltd. | 44657-6 |
| Negative control virus | GeneChem Co., Ltd. | LVC0N335 |
| CON335(Ubi-MCS-3FLAG- | | |
| CBh-gcGFP-IRES-puromycin) | | |
| Cell Cryopreservation Solution | Corning | 88-702-CB |
| MTT | Solarbio | M8180 |
| H₂O₂ | Jiangxi Caoshanhu | 20190103 |
| | disinfection supplies | |
| | Co., Ltd. | |
| ROS detection kit | Beyotinne | S0033 |

2. Experimental Procedure and Results

1. Isolation, Culture and Induced Differentiation of Porcine Skeletal Muscle Satellite Cells

1.1 Isolation and Culture of Porcine Skeletal Muscle Satellite Cells

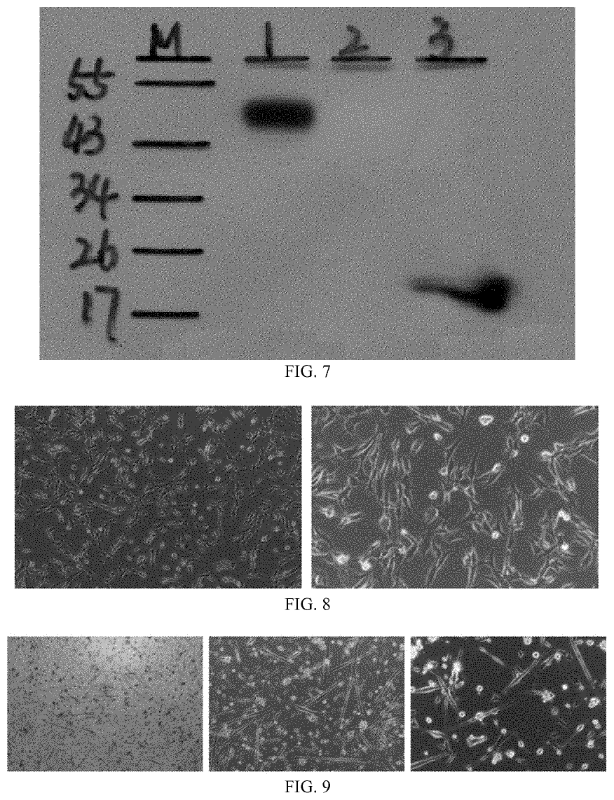

The piglets was bled to death at the neck artery, then the uterine tissue was taken out, placed in PBS containing P/S, and the piglet was disinfected with 75% alcohol. The thigh skeletal muscle was separated under aseptic conditions and placed in PBS containing a secondary antibody. The thigh skeletal muscle was rinsed repeatedly for 3-5 times, transferred to a petri dish, minced into 1 mm³-size tissue blocks with an ophthalmological scissor. The minced tissue blocks were transferred to a 50 mL centrifuge tube, 0.25% Trypsin was added and digested at 37° C. for 30 min in water bath and under vibration, and then a complete medium was used to neutralize the digestion fluids. The mixture of the digested tissue was filtered through a 100 μm mesh, the filtrate was collected and centrifuged at 1500 rpm for 10 min. After the completion of centrifugation, the supernatant was discarded; the precipitate was re-suspended with culture medium, transferred to a T-25 culture flask. The 1-25 culture flask was placed into an incubator, and the culture medium was changed every 24 hours. The cultured cells are shown in FIG. 8, which shows that the cell culture was successful.

1.2 Induced Differentiation of Porcine Skeletal Muscle Satellite Cells

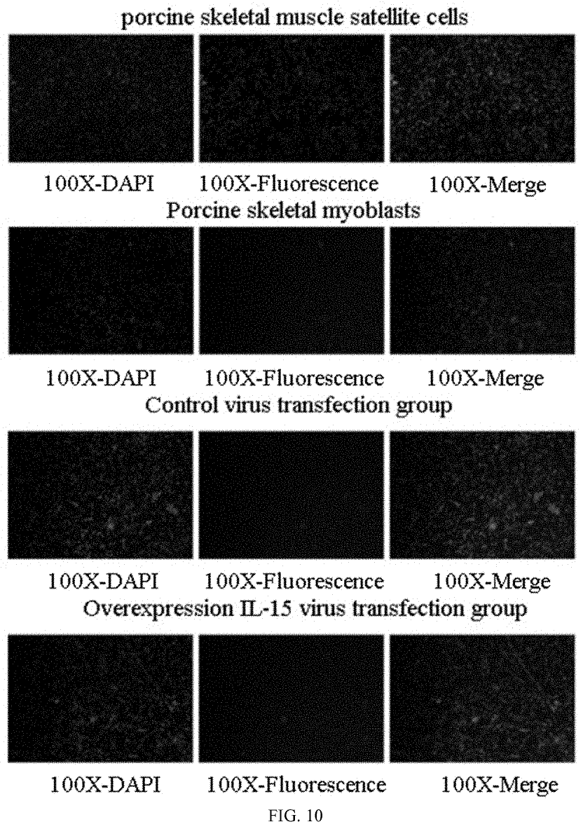

The second-generation porcine skeletal muscle satellite cells were inoculated in a culture flask padded with L-polylysine. When the cell fusion degree was about 50-60%, differentiation medium DMEM (low sugar)+2% goat Serum was added and the medium was changed on the third day of the culturing. After culturing for a total of 5 days, growth of the cells was observed. The cultured cells are shown in FIG. 9, which shows that the cells had successfully differentiated and the porcine skeletal myoblasts were thus obtained.

1.3 Transfection with Lentivirus Overexpressing IL-15

The differentiated myoblasts were inoculated into a 6-well plate, the number of cells per well was about 1×10⁵ cells. The medium was changed when cells were cultured to wall adherence. One milliliter of complete medium was added, and then 20 μL of control lentivirus and lentiviral overexpression (MOI=100) were added respectively, the resulting culture was mixed well and continued to culture for 72 h transfection.

1.4 Immunofluorescence Assay

Porcine skeletal muscle satellite cells, differentiated porcine skeletal myoblasts, porcine skeletal myoblasts transfected with control lentivirus, and porcine skeletal myoblasts transfected with overexpression lentivirus were measured. Specific steps are as follows:

(1) Round Coverslip

Three round coverslips in a 24-well plate, 1 mL of culture medium was added to each well, and 0.2×10⁶ cells/well was added. The resulting round coverslips were placed in the incubator and incubated for 2 h or overnight.

(2) Immobilization

After the cells are attached to the round coverslips, the culture medium was aspirated, and the round coverslips were washed with PBS 1 time and 4% PFA was added at 4° C. and immobilization was carried out for 30 min. The round coverslips were washed with PBS 3 times, and 5 min each time. Alternatively, the round coverslips may be allowed to stand at 4° C. overnight without aspirating the PBS for the last time.

(3) Permeabilization and Blocking

The round coverslips was get rid of water and placed on a Petri dish support.

Preparation of blocking solution for the round coverslips: 0.5% Trition X-100 mixed with PBS in a ratio of 1:1, with additional 10% serum.

Fifty microliter of permeabilization wash buffer was dripped on a waterproof membrane, and the surface having cells was covered and kept for 2 hours.

(4) Incubation of Primary Antibody

Preparation of primary antibody (α-SMA): antibody was diluted with PBS in a ratio of 1:100.

After the permeabilization and blocking, 50 μL of primary antibody was applied to a waterproof membrane (in a humidified chamber), and the round coverslips (the surface having cells) was covered and kept at 4° C. (for up to one week).

(5) Incubation of Secondary Antibody

Preparation of secondary antibody (CoraLite594-conjugated Goat Anti-Rabbit IgG (H+L)): antibody was diluted with PBS in a ratio of 1:500.

After the secondary antibody was incubated at room temperature for 2 hours in the dark, the secondary antibody was washed with PBS 3 times and 5 min for each time. Then the secondary antibody was stained with DAPI (DAPI:PBS =1:1000) for 5 min and washed with PBS 3 times and 5 min for each time.

(6) Embedding

One drop of Fluoromount-G was dripped on each round coverslips, and the surface having cells was covered for observation.

The results of the fluorescence measurement are shown in FIG. 10.

1.5 Determination of Cell Cycle

The cells were digested with EDTA-free trypsin, and centrifuged at 1000 r/min for 5 min, cells were collected, washed with PBS, and immobilized overnight with at 75% pre-cooled ethanol. The resulting mixture was centrifuged at 1000 r/min for 10min and ethanol was discarded. The cells were incubated in a staining solution containing 100 μg/mL of PI and 200 μg/mL of RNAase and incubated for 30 min. The apoptotic cell peak was detected by flow cytometry, and the fluorescence signals were processed by cell cycle analysis software Multicycle in a flow cytometer.

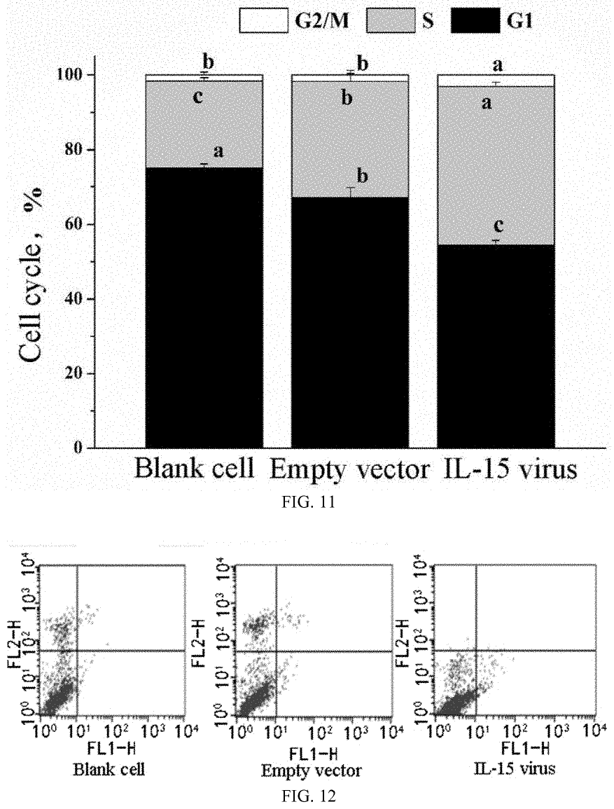

The test results are shown in FIG. 11. After transfection of IL-15, the proportion of cells in G1 phase decreased significantly, and the proportion of cells in S phase and G2/M phase increased significantly. It is indicated that IL-15 could promote the division and proliferation of myoblasts.

1.6 Detection of Apoptosis

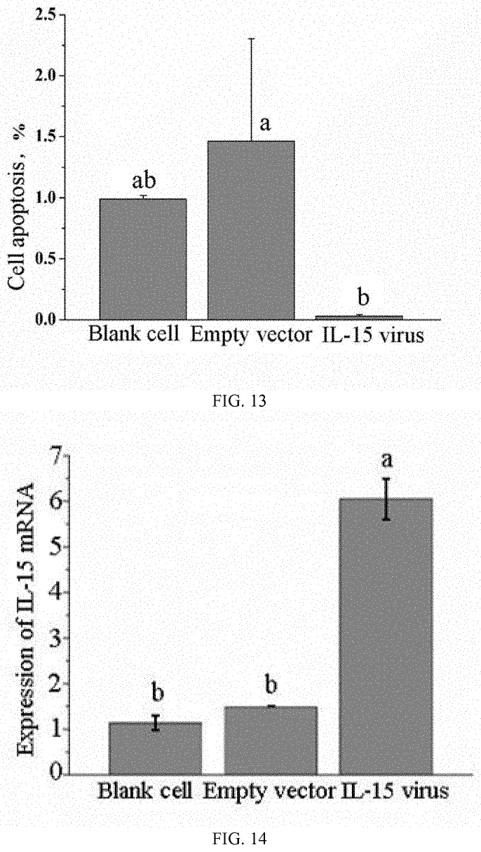

An Annexin V-FITC Apoptosis Detection Kit (Tongjin Institute of Chemistry, Japan, lot number VN665) was used for apoptosis detection. The cells were digested with trypsin without EDTA and centrifuged at 1000 r/min for 5 min. 1×AnnexinV binding buffer was added after the cells were resuspended. 5 μL of Annexin V-FITC, 5 μL of PI were added and mixed well, the resulting mixture was incubated at room temperature in dark for 15 min, and the cells were detected by flow cytometry, the detection results are shown in FIG. 12 and FIG. 13. After the cells were transfected with IL-15, the apoptosis rate of myoblasts decreased significantly. It shows that IL-15 can inhibit the apoptosis of myoblasts.

1.7 Measurement of Cell Gene Expression

1.7.1 Materials and Reagents

The cells were digested with EDTA-free trypsin and centrifuged at 1000 r/min for 5 min. The cells were collected for detection of IL-15 gene expression. The instruments and reagents used are shown in Table 8.

TABLE 8
Instruments and reagents for use

| Reagent/Instrument | Manufacturer | Product No. |
|---|---|---|
| TRNzol total RNA | TIANGEN BIOTECH | DP405-02 |
| extraction reagent | (BEIJING) CO., LTD. | |
| PrimeScript ™ RT reagent | TaKaRa (Dalian) | RR047B |
| Kit with gDNA Eraser | | |
| SYBR ® Premix Ex Taq ™ | TaKaRa (Dalian) | RR82LR |
| II (Tli RNaseH Plus), | | |
| ROX plus | | |
| DL2,000 DNA Marker | TaKaRa (Dalian) | 3427Q |
| primer synthesis | Invitrogen | |
| Vortex oscillator | Haimen Kylin-Bell Lab | QL-902 |
| | Instruments Co., Ltd. | |
| Centrifuge | Eppendorf | Centrifuge |
| | | 5415D |
| Spectrophotometer | Therno scientific | NANODROP |
| | | 2000 |
| Gel imaging system | Shanghai Tanon | Tanon 1600 |
| | Technology Co., Ltd. | |
| Fluorescence Quantitative | Applied Biosystems | ABI7500 |
| PCR Instrument | | |

The primers used in Real Time PCR to detect the target gene are shown in Table 9 (SEQ ID NO.5-SEQ ID NO. 8). The following primers were synthesized by Beijing Invitrogen Company.

TABLE 9
Primer information used to measure gene expression

| | | Product |
|---|---|---|
| | Primer | size |
| Primer name | sequence (5′ to 3′) | (bp) |
| Upstream primer | GCATCCAGTGCTACTTGTGTR | 118 |
| for IL15 | | |
| Downstream primer | TGCCAGGTTGCTTCTGTTTT | |
| for IL15 | | |
| Upstream primer | TGCGGGACATCAAGGAGAAG | 216 |
| for ACTIN | | |
| Downstream primer | AGTTGAAGGTGGTCTCGTGG | |
| for ACTIN | | |

1.7.2 Experimental Method

1.7.2.1 Extraction and Quality Testing of Sample RNA

A TRNzol total RNA extraction reagent was use for extraction of sample RNA, and the experimental operation was carried out according to the product instructions.

The concentration and purity were measured by a UV absorption method. An ND-2000 ultraviolet-visible spectrophotometer was used to measure the concentration and purity of RNA, and the spectrophotometer was adjusted to zero with a DEPC solution which was used to dissolve RNA before measurement. The readings at 260 nm and 280 nm were measured to calculate the concentration and purity of the RNA sample.

RNA concentration was calculated as follows: a reading value of 1 at 260 nm was equivalent to 40 ng RNA/μL. The formula for calculating the sample RNA concentration was: A260×40 ng/μL. In this Example, it was measured A 260=65.003. Upon calculation, RNA concentration was 2600.12 ng/μL, the remaining total amount of RNA excluding the consumed test sample was 19 μL×2600.12 ng/UL=49.4 μg.

RNA purity was judged through the ratio of A260/A 280, and the ratio ranged from 1.8 to 2.1. Even if the ratio exceeded this range, RNA samples could also be used in some common experiments such as Northern hybridization, RT-PCR and RNase protection experiments. The ratio of A260/A280 measured in this embodiment was 2.0.

1.7.2.3 Denaturing Agarose Gel Electrophoresis

One gram of agarose was dissolved in 72 ml of water, cooled to 60° C., and 10 ml of 10×MOPS electrophoresis buffer and 18 ml of 37% formaldehyde solution (12.3 M) were added.

Calculated based on the final concentration, 10×MOPS electrophoresis buffer contained 0.2 M MOPS (pH 7.0), 0.02M sodium acetate, and 0.01M EDTA.

Gel plates were filled and sample wells reserved for addition of at least 25 μL of solution. After gelation, the combs were removed, and the gel plates were placed in the electrophoresis tank, and a sufficient amount of 1×MOPS electrophoresis buffer was added to cover the gel surface by a few millimeters.

Into 1 μg of RNA was added 1 volume of the formaldehyde loading stain and EB was added in the formaldehyde loading stain to a final concentration of 10 μg/ml. The mixture was heated to 70° C. and incubated for 5 minutes to denature the sample. The sample was loaded into the gel well, and electrophoresis was conducted at a voltage of 5-6V/cm until the bromophenol blue indicator entered the gel by at least 2-3 cm. Observation was performed under transmitted UV light, the results showed that the RNA sample had a high purity and could be used in subsequent experiments.

1.7.2.4 Sample Detection by Real Time PCR

The purified RNA samples were reverse transcribed to cDNA using PrimeScript™ RT reagent Kit with gDNA Eraser. The resulting cDNA samples were tested by Real Time PCR, and the specific steps were included:

(1) Preparation of reaction system: Real Time PCR reaction systems were separately prepared for all cDNA samples. Realtime PCR reaction systems each had a total volume of 18 μL, specifically containing 10 μL of 2×Master Mix, 0.5 μL, of 10 μM PCR specific primers F, 0.5 μL of 10 μM of PCR specific primers R, and water was added to a total volume of 18 μL. The tube was flicked at the bottom to mix the solution, and centrifuged briefly at 5000 rpm.

(2) Sample loading: 18 μL of mixed solution was added to each corresponding well of a 96-well PCR plate, and 2 μL of cDNA was added. A sealing film was attached carefully and the tube was mixed by brief centrifugation. The well prepared PCR plate was placed on ice before setting the PCR procedure.

(3) PCR reaction: proceed as following procedure: 95° C., 30 s; 40 PCR cycles (95° C., 5 s; 60° C., 40 s (fluorescence was collected)). In order to establish the melting curve of the PCR product, the PCR product was slowly heat from 60° C. to 99° C. (the instrument automatically implemented a Ramp rate of 0.05° C./s) as per a procedure of 95° C., 10 s; 60° C., 60 s; and 95° C., 15 s) after the amplification reaction is completed.

1.7.3 Experimental Results

The target gene and internal reference gene of each sample were subjected to Real Time PCR reaction, and each sample was tested in 3 replicate wells. The data was analyzed by 2^{-ΔΔ}^{^{CT}} method.

The results are shown in FIG. 14. After transfection with lentivirus, expression of IL-15 gene in myoblasts increased significantly. It is showed that the lentivirus overexpressing IL-15 was successfully constructed.

1.8 Measurement of Cell Protein Expression

The cells were digested with EDTA-free trypsin and centrifuged at 1000 r/min for 5 min. The cells were collected and the IL-15 protein expression was measured.

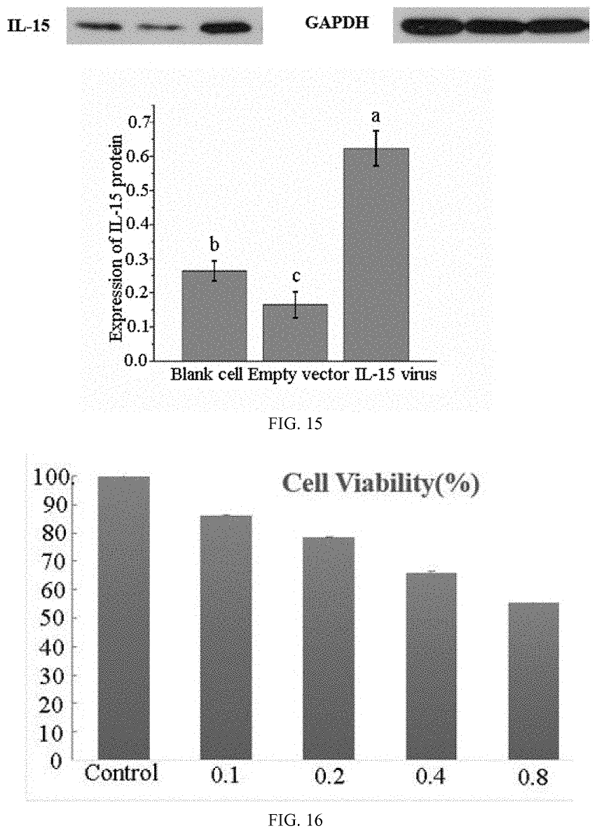

The results are shown in FIG. 15. It is showed that protein expression from IL-15 in myoblasts increased significantly after transfecting lentivirus.

### Example 3

Effect of IL-15 on oxidative damage of porcine skeletal myoblasts

1. Establishment of Oxidative Stress Model

The MTT method was used to measure cell viability.

(1) The porcine skeletal myoblasts was inoculated in a 96-well plate in a number of 1×10⁴/well and the plate was incubated at 37° C. for 24 hours in an incubator filled with 5% of CO_{2.}

(2) The culture liquid was removed and a culture medium containing 0.1, 0.2, 0.4 and 0.8 mM H₂O₂ respectively, and a culture medium without addition of H₂O₂ as control. Five replicates were set in each group, and the cells were continued to culture for further 1.5 hours.

(3) The culture medium was removed and washed with PBS 2 times.

(4) To each well was added 20 μL of 5 g/L of MTT solution, culturing was continued for another 4 hours.

(5) The supernatant was removed, and 150 μL, of DMSO was added to each well and shaken for 10min;

(6) OD values for each well were measured by an enzyme-labeled instrument at a wavelength of 570 nm.

The results are shown in FIG. 16. It is shown that when the oxidative stress model was established, the cell survival rate was usually in the range of 50% -70%. If the cell survival rate was too high, no significant oxidative damage was caused, and if the survival rate was too low, irreversible damage was caused. In both cases, it was not conducive to the establishment of an oxidative stress model. Therefore, 0.1 mM and 1.5 h were used as the concentration and time in the treatment of the H₂O₂ oxidative stress model, respectively.

2. The Effect of IL-15 on the Oxidative Damage of Porcine Skeletal Myoblasts

Porcine skeletal muscle satellite cells were primarily cultured, when the cell fusion degree was about 50-60%, differentiation culture medium (low-sugar DMEM medium+2% goat serum) was added to induce differentiation of cells for 5 days. Then the differentiated myoblasts were transfected with an empty vector or GV492-IL-15 recombinant lentivirus for cell treatment. Seventy two hours later, cells were treated with 0.1 mM H₂O₂ for 1.5 h to form three experimental treatment groups of blank cells+H₂O₂ group, empty vector+H₂O₂ group and GV492-IL-15 transfected recombinant lentivirus +H₂O₂ group.

1. Measurement of Cell Cycle

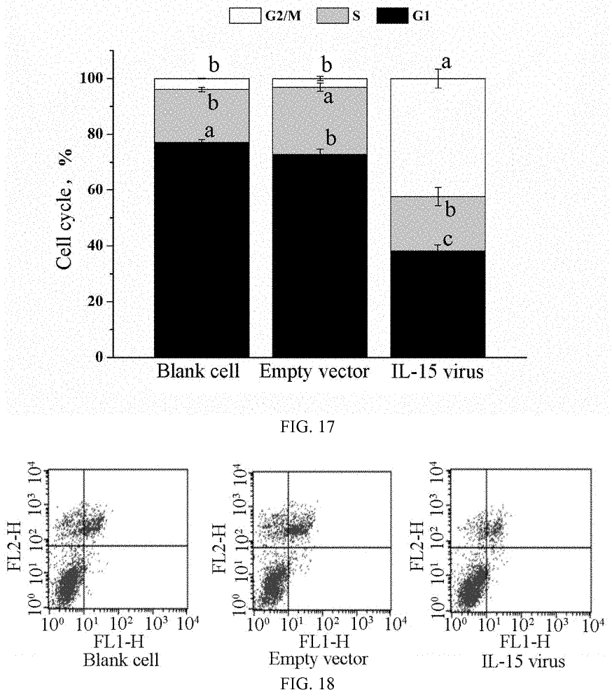

Measurement cell cycle was performed according to the method of Example 2. As shown in FIG. 17, under oxidative stress conditions, the ratio of cells in G1 phase after IL-15 transfection significantly decreased, and the ratio of cells in G2/M phase increased significantly. Compared with the empty vector group, the proportion of S-phase cells decreased significantly, while compared with the blank cells, the difference was not significant. It is shown that IL-15 can promote the division and proliferation of myoblasts.

2. Measurement of Cell Apoptosis

2.1 Measurement by Flow Cytometry

The cells were digested with EDTA-free trypsin and centrifuged at 1000 r/min for 5 min. s1×Annexin V binding buffer was added after the cells were resuspended. Five microliter of Annexin V-FITC, 5 μL of PI were added and mixed well, the resulting mixture was incubated at room temperature in dark for 15 min, and then the cells were measured by flow cytometry.

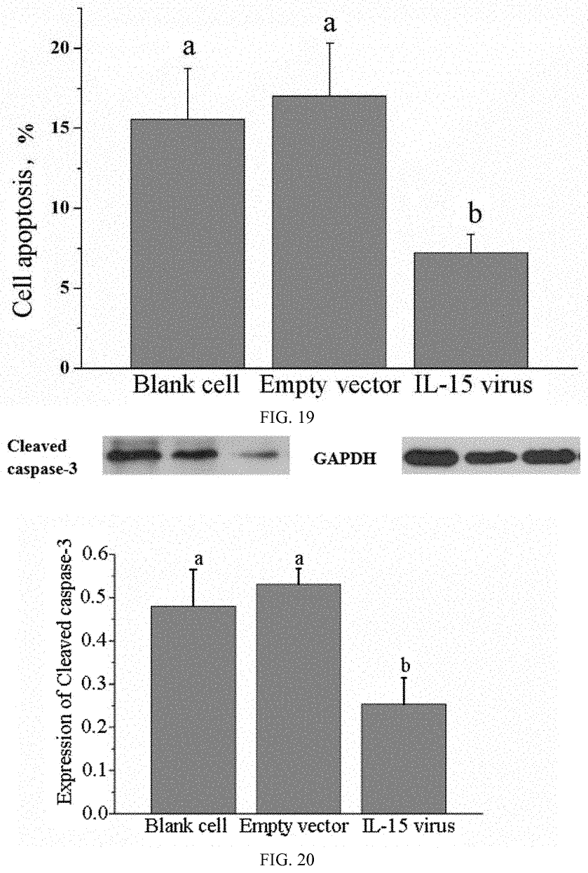

The test results are shown in FIG. 18 and FIG. 19. It can be seen that the proportion of apoptosis after transfection of IL-15 was significantly reduced. It is shown that IL-15 can reduce the apoptosis rate of cells under oxidative stress.

2.2 Measurement by Western Blot

The changes of cell apoptosis was reflected by measurement the expression profile of activated caspase-3 protein in the cells. Cleavedcaspase-3 antibody was purchased from Immunoway Company, catalog number YM3431, dilution ratio 1:500.

The results are shown in FIG. 20. Oxidative stress significantly increased the apoptotic rate of myoblasts, while transfection of IL-15 lentivirus could significantly decrease the apoptotic rate of cells, which confirmed that IL-15 can relieve oxidative damage by inhibiting cell apoptosis.

3. Measurement of ROS in Cell

The cells treated with H₂O₂ were digested with cold Tyrode buffer and washed twice, and resuspended with 500 μL of Tyrodebuffer. Then 10 μM DCFH-DA (Sigma Corporation) was added, and cells were incubated at 37° C. for 30 min in dark, centrifuged, and the supernatant was discarded, and then washed with the buffer twice gently. The cells were resuspended after the supernatant was decanted, and the fluorescence intensity in each group was measured by flow cytometry, respectively.

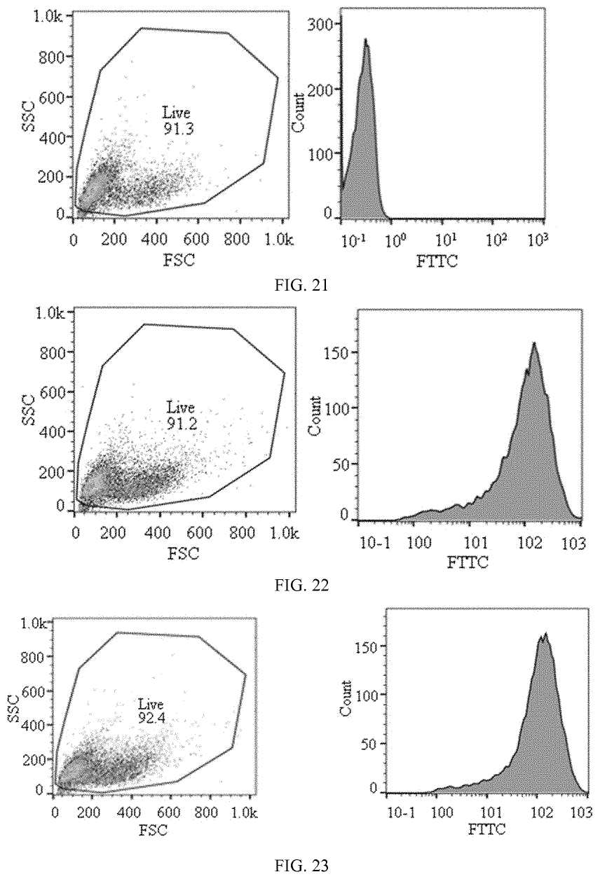

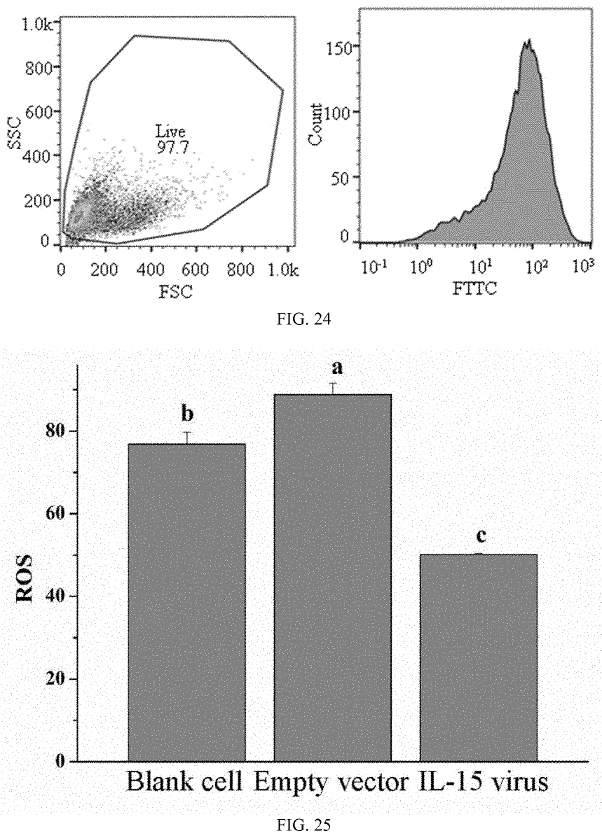

The results shown in FIG. 21-FIG. 25, it is shown that oxidative stress could significantly increase the ROS content in the cells. Treatment of lentivirus by transfection of IL-15 could significantly reduce the ROS content in cells. It is shown that IL-15 can inhibit the oxidative damage of myoblasts to a certain extent.

4. Secretion of IL-15 in Cells

The treated cell culture supernatant was collected, and the secretion of IL-15 was measured with an ELISA kit (R&D Systems).

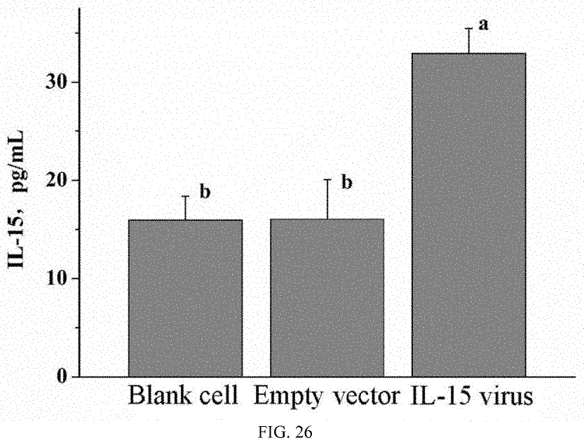

The results in FIG. 26, the under oxidative stress conditions, secretion of IL-15 in cells increased significantly after transfection of IL-15 in lentivirus, indicating that IL-15 could alleviate cell damage caused by oxidative stress.

The above-mentioned embodiments merely describe the preferred embodiments of the present disclosure, and are not intended to limit the scope of the present disclosure. Those of ordinary skill in the art can make various variations and improvements to the technical solutions of the present disclosure, without departing from the design spirit of the present disclosure. These variations and improvements should fall within the protection scope set forth by the appended claims of the present disclosure.

The application hereby incorporates by reference the Sequence Listing titled “BGI05-005-US_Sequence_Listing.txt”, created on Feb. 15, 2022, having a file size of 3 KB.

What is claimed is:

1. Method for overexpressing IL-15 in porcine skeletal myoblasts, wherein the method comprises steps of: transforming IL-15 into a lentiviral vector, and transforming the lentiviral vector into porcine skeletal myoblasts to promote the porcine skeletal myoblasts to overexpress IL-15.

2. The method according to claim 1, wherein the lentiviral vector is GV492 lentiviral expression vector.

3. Use of overexpression of IL-I5 in reducing apoptosis of porcine skeletal myoblasts.

4. Use of overexpression of IL-15 in enhancing the ability of porcine skeletal myoblasts to resist oxidative damage.

5. Use of overexpression of IL-15 in promoting division and proliferation of porcine skeletal myoblasts.


SEQUENCE LISTING (9 sequences)

SEQ ID NO: 1 - DNA, 50 bp, Artificial Sequence
  Forward primer for PCR
aggtcgctctagaggatcccgccaccatgagaattttgaaaccatgtttg

SEQ ID NO: 2 - DNA, 39 bp, Artificial Sequence
  Reverse primer for PCR
tccttgtagtccataccagaagggttgatgaacatttgc

SEQ ID NO: 3 - DNA, 23 bp, Artificial Sequence
  Forward primer for PCR  identification
gggtcaatatgtaattttcagtg

SEQ ID NO: 4 - DNA, 22 bp, Artificial Sequence
  Reverse primer for PCR identification
ccttatagtccttatcatcgtc

SEQ ID NO: 5 - DNA, 21 bp, Artificial Sequence
  Upstream primer for measuring gene expression of IL15
gcatccagtgctacttgtgtr

SEQ ID NO: 6 - DNA, 20 bp, Artificial Sequence
  Downstream primer for measuring gene expression of IL15
tgccaggttgcttctgtttt

SEQ ID NO: 7 - DNA, 20 bp, Artificial Sequence
  Upstream primer for measuring gene expression of ACTIN
tgcgggacatcaaggagaag

SEQ ID NO: 8 - DNA, 20 bp, Artificial Sequence
  Downstream primer for measuring gene expression of ACTIN
agttgaaggtggtctcgtgg

SEQ ID NO: 9 - protein, 8 aa, Artificial Sequence
  Sequence of hydrophilic peptide consisting of eight amino acids
DYKDDDDK